(19) United States
(12) Patent Application Publication
(10) Pub. No.: US 2025/0057585 A1
(43) Pub. Date: Feb. 20, 2025

(54) ELECTROSURGICAL TRANSSEPTAL ASSEMBLY WITH PROTRUSION ALERT

(71) Applicant: Boston Scientific Medical Device Limited, Galway (IE)

(72) Inventors: Jackie Leung, Richmond Hill (CA); Matthew MacDonald, Calgary (CA)

(21) Appl. No.: 18/805,811

(22) Filed: Aug. 15, 2024

Related U.S. Application Data
(60) Provisional application No. 63/519,730, filed on Aug. 15, 2023.

(51) Int. Cl. A61B 18/14; A61B 18/00; A61B 18/12; A61B 90/00

(52) U.S. Cl. CPC A61B 18/14; A61B 18/1206; A61B 90/08; A61B 2018/00184; A61B 2018/00351; A61B 2018/1253; A61B 2018/144; A61B 2018/1475; A61B 2090/0811

(57) ABSTRACT

A transeptal surgical system to puncture an atrial septum within a heart of a patient is disclosed. The transseptal surgical system includes an electrosurgical device configured to couple to a radiofrequency (RF) energy source and an electroanatomical mapping system coupled to the electrosurgical device. The electrosurgical device includes a delivery component having a delivery component electrode. The electrosurgical device also includes a crossing member within the lumen and coupled to the RF energy source having a crossing member electrode to deliver the RF energy. The mapping system detects a position of the delivery component electrode and the crossing member electrode, determines a distance between the crossing member electrode and the delivery component electrode, and generates an alert if the crossing member electrode is extended from the delivery component electrode at a specified protrusion spacing.

### CROSS REFERENCE TO RELATED APPLICATIONS

This application claims priority to U.S. Provisional Patent Application No. 63/519,730 entitled “ELECTROSURGICAL TRANSSEPTAL ASSEMBLY WITH PROTRUSION ALERT,” filed Aug. 15, 2023, which is incorporated herein by reference in its entirety.

### TECHNICAL FIELD

The present disclosure relates to medical devices and systems for use in percutaneous or interventional procedures including surgery. More specifically, this disclosure relates to electrosurgical devices, assemblies, and systems that provide for cutting or puncturing of bodily tissues such as the atrial septum with an electrode.

### BACKGROUND

Catheters are often used to provide general access into a patient's body using minimally invasive techniques. In some examples, a catheter can be used to create a channel through a region of the body. One such example is a transseptal puncture in a cardiac procedure. The left atrium is a difficult cardiac chamber to access reach percutaneously. Although the left atrium can be reached via the left ventricle and mitral valve, the catheter is manipulated through two U-turns, which can be cumbersome. the transseptal puncture is a technique of creating a small surgical passage through the atrial septum, or wall in the heart between the left and right atrium, through which a catheter can be fed. The atrial septum is punctured and dilated via tools. The transseptal puncture permits a direct route to the left atrium via the intra-atrial septum and systematic venous system. Increasing larger and complex medical devices can be passed into the right atrium. Historically, the technique was used exceptionally for mitral valvuloplasty and ablation in the left heart. Today, the increased interest in catheter ablation and its application in many other procedures has meant the transseptal puncture is a routine technique for interventional cardiologists and cardiac electrophysiologists.

Transseptal punctures can be performed with the aid of guidewires having electrodes energized with a suitable power source such as an electrically coupled power generator in a manner like other electrosurgical devices. Typical electrosurgical devices apply an electrical potential difference or a voltage difference between an active electrode and a return electrode on a patient's grounded body in a monopolar arrangement or between an active electrode and a return electrode on the device in bipolar arrangement to deliver electrical energy to the area where tissue is to be affected. Electrosurgical devices pass electrical energy through tissue between the electrodes to cut or puncture tissue with plasma formed on the energized electrode. Tissue that contacts the plasma experiences a rapid vaporization of cellular fluid to produce a cutting effect. Electrical energy can be applied to the electrodes either as a train of high frequency pulses or as a continuous signal typically in the radiofrequency (RF) range to perform the cutting or puncturing techniques.

Visualization and guidance systems are routinely used in electrophysiology procedures, including procedures that employ transseptal punctures, to facilitate the delivery of catheters and other devices. Although the evolution of X-ray technologies and fluoroscopic guidance has enabled highly accurate imaging of complex anatomies, such imaging techniques expose patients and clinicians to radiation. Newer X-ray technologies and optimized fluoroscopic exposure parameters have been noted to reduce image quality and provide difficulties in visualization for less experienced clinicians. Emerging non-fluoroscopic visualization technologies for electrophysiological procedures including transseptal punctures include three-dimensional electroanatomical mapping (EAM) and two-dimensional intracardiac echocardiography (ICE) or transesophageal echocardiography (TEE). Limited visualization of the transseptal apparatus on such techniques has been associated with reduced transseptal puncture success, and clinicians will often switch to fluoroscopic guidance in such circumstances. In the absence of adequate visualization of the transseptal apparatus with alternative visualization techniques, fluoroscopy continues to be used in transseptal puncture.

### SUMMARY

In an Example 1, a transeptal surgical system configured to puncture an atrial septum within a heart of a patient, the transseptal surgical system comprising: an electrosurgical device configured to couple to a radiofrequency (RF) energy source, the electrosurgical device comprising a delivery component having an elongate shaft defining a longitudinally extending lumen and a delivery component distal tip having a delivery component electrode, and a crossing member adapted to be disposed within the lumen and coupled to the RF energy source, the crossing member having a crossing member distal tip extendable from the lumen, the crossing member distal tip having a crossing member electrode adapted to deliver the RF energy; and an electroanatomical mapping system coupled to the electrosurgical device, the electroanatomical mapping system having a tracking component configured to detect a position of the delivery component electrode and the crossing member electrode, determine a distance between the crossing member electrode and the delivery component electrode, and generate an alert if the crossing member electrode is extended from the delivery component electrode at a specified protrusion spacing.

In an Example 2, the transeptal surgical system of Example 1, wherein the delivery component is a dilator having a tapered distal portion, the tapered distal portion including the delivery component distal tip.

In an Example 3, the transseptal surgical system of any of Examples 1 and 2, wherein the delivery component electrode is an annular electrode around the delivery component distal tip

In an Example 4, the transeptal surgical system of any of Examples 1-3, and further comprising an RF generator to provide the RF energy source.

In an Example 5, the transseptal surgical system of Example 4, and further comprising a switch mechanically coupled to the electrosurgical device, the RF generator, and the electroanatomical mapping system, the switch configured to selectively electrically couple the electrosurgical device to one of the RF generator and the electroanatomical mapping system.

In an Example 6, the transseptal surgical system of Example 5, wherein the switch is configured to selectively electrically couple the crossing member electrode to one of the RF generator and the electroanatomical mapping system.

In an Example 7, the transeptal surgical system of any of Examples 1-6, wherein the crossing member is adapted to deliver the RF energy in a monopolar mode.

In an Example 8, the transeptal surgical system of any of Examples 1-7, wherein the crossing member is configured as a guidewire.

In an Example 9, the transeptal surgical system of any of Examples 1-8, wherein the crossing member distal tip is extendable from the delivery component distal tip such that the delivery component is fully retractable over the crossing member.

In an Example 10, the transeptal surgical system of any of Examples 1-9, and further comprising determining whether the detected position of the crossing member electrode lies on an axis of the delivery component electrode, and wherein the alert is generated if the crossing member electrode is extended from the delivery component electrode at the specified protrusion spacing and the detected position of the crossing member electrode lies on the axis of the delivery component electrode.

In an Example 11, the transeptal surgical system of Example 10, wherein the delivery component includes an orientation electrode, and the tracking component is configured to detect a position of the orientation electrode and define the axis of the delivery component electrode to include the position of the delivery component electrode and the orientation electrode.

In an Example 12, the transeptal surgical system of any of Examples 10-11, wherein the orientation electrode is disposed proximal to the delivery component electrode along the elongate shaft.

In an Example 13, the transeptal surgical system of any of Examples 10-12, wherein delivery component includes a dilator/sheath assembly and the orientation electrode is disposed on the dilator sheath assembly.

In an Example 14, the transeptal surgical system of any of Examples 10-13, wherein the orientation electrode includes a plurality of orientation electrodes.

In an Example 15, the transeptal surgical system of any of Examples 10-14, wherein the orientation electrode is an annular ring electrode.

In an Example 16, a transeptal surgical system configured to puncture an atrial septum within a heart of a patient, the transseptal surgical system comprising: an electrosurgical device configured to couple to a radiofrequency (RF) energy source, the electrosurgical device comprising a delivery component having an elongate shaft defining a longitudinally extending lumen and a delivery component distal tip having a delivery component electrode, and a crossing member adapted to be disposed within the lumen and coupled to the RF energy source, the crossing member having a crossing member distal tip extendable from the lumen, the crossing member distal tip having a crossing member electrode adapted to deliver the RF energy; and an electroanatomical mapping system coupled to the electrosurgical device, the electroanatomical mapping system having a tracking component configured to detect a position of the delivery component electrode and the crossing member electrode, determine a distance between the crossing member electrode and the delivery component electrode, and generate an alert if the crossing member electrode is extended from the delivery component electrode at a specified protrusion spacing.

In an Example 17, the transeptal surgical system of Example 16, wherein the delivery component is a dilator having a tapered distal portion, the tapered distal portion including the delivery component distal tip.

In an Example 18, the transeptal surgical system of Example 16, and further comprising an RF generator to provide the RF energy source.

In an Example 19, the transseptal surgical system of Example 18, and further comprising a switch mechanically coupled to the electrosurgical device, the RF generator, and the electroanatomical mapping system, the switch configured to selectively electrically couple the electrosurgical device to one of the RF generator and the electroanatomical mapping system.

In an Example 20, the transseptal surgical system of Example 19, wherein the switch is configured to selectively electrically couple the crossing member electrode to one of the RF generator and the electroanatomical mapping system.

In an Example 21, the transeptal surgical system of Example 16, wherein the crossing member is adapted to deliver the RF energy in a monopolar mode.

In an Example 22, the transeptal surgical system of Example 16, wherein the crossing member is configured as a guidewire.

In an Example 23, the transeptal surgical system of Example 16, wherein the crossing member distal tip is extendable from the delivery component distal tip such that the delivery component is fully retractable over the crossing member.

In an Example 24, the transeptal surgical system of Example 16, wherein the delivery component includes an orientation electrode, and the tracking component is further configured to detect a position of the orientation electrode, define an axis of the delivery component to include the position of the delivery component electrode and the orientation electrode, and generate the alert if the crossing member electrode is extended from the delivery component electrode at the specified protrusion spacing and the detected position of the crossing member electrode lies on the axis of the delivery component.

In an Example 25, the transeptal surgical system of Example 24, wherein the orientation electrode is disposed proximal to the delivery component electrode along the elongate shaft.

In an Example 26, an electroanatomical mapping system for use with an electrosurgical device to guide a surgical puncture of an atrial septum within a heart of a patient, the electrosurgical device comprising a delivery component having an elongate shaft defining a longitudinally extending lumen and a delivery component distal tip having a delivery component electrode, and a crossing member adapted to be disposed within the lumen, the crossing member having a crossing member distal tip extendable from the lumen, the crossing member distal tip having a crossing member electrode adapted to deliver a source of RF energy, the electroanatomical mapping system comprising: memory to store a set of instructions; and a processor to execute the set of instructions to detect a position of the delivery component electrode and the crossing member electrode, determine a distance between the crossing member electrode and the delivery component electrode, and generate an alert if the crossing member electrode is extended from the delivery component electrode at a specified protrusion spacing.

In an Example 27, the electroanatomical mapping system of Example 26, wherein the set of instructions further includes instructions to receive the specified protrusion spacing from a user input.

In an Example 28, the electroanatomical mapping system of Example 26, wherein the instructions to generate the alert include instructions to generate the alert based on a congruence of the position of the delivery component electrode and the crossing member electrode and the specified protrusion spacing.

In an Example 29, the electroanatomical mapping system of Example 26, wherein instructions to detect the position of the delivery component electrode and the crossing member electrode include instructions to superimpose the position of the delivery component electrode and the crossing member electrode on a graphical representation of the heart.

In an Example 30, the electroanatomical mapping system of Example 29, wherein instructions to generate the alert include instructions to highlight a graphical illustration of the crossing member electrode extended from the delivery component electrode superimposed on the graphical representation of the heart.

In an Example 31, the electroanatomical mapping system of Example 26, wherein the set of instructions further includes instructions to determine the specified protrusion spacing from a set of received surgical parameters related to the electrosurgical device and the heart.

In an Example 32, a process to guide an electrosurgical device in a surgical puncture of an atrial septum within a heart of a patient, the electrosurgical device comprising a delivery component having an elongate shaft defining a longitudinally extending lumen and a delivery component distal tip having a delivery component electrode, and a crossing member adapted to be disposed within the lumen, the crossing member having a crossing member distal tip extendable from the lumen, the crossing member distal tip having a crossing member electrode adapted to deliver a source of RF energy, the process comprising: detecting a position of the delivery component electrode and the crossing member electrode with an electroanatomical mapping system electrically coupled to the electrosurgical device; determining a distance between the crossing member electrode and the delivery component electrode with the electroanatomical mapping system; and generating an alert with the electroanatomical mapping system if the crossing member electrode is extended from the delivery component electrode at a specified protrusion spacing.

In an Example 33, the process of Example 32, wherein generating an alert is based on a congruence of the position of the delivery component electrode and the crossing member electrode and the specified protrusion spacing from a user input.

In an Example 34, the process of Example 33, and further comprising superimposing the position of the delivery component electrode and the crossing member electrode on a graphical representation of the heart in a display device.

In an Example 35, the process of Example 34, wherein instructions to generate the alert include instructions to highlight a graphical illustration of the crossing member electrode extended from the delivery component electrode superimposed on the graphical representation of the heart.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic diagram illustrating an example electrosurgical system for treating a patient, such as a heart or the vasculature of a patient, including an electrosurgical generator, an electrosurgical device, and an electroanatomical mapping (EAM) system.

FIG. 2 is a schematic diagram illustrating an embodiment of electrosurgical device for use in the example electrosurgical system of FIG. 1.

FIG. 3 is a schematic diagram illustrating a controller of the EAM system for use in the electrosurgical system of FIG. 1.

FIG. 4 is a block diagram illustrating an embodiment of a process for use with the EAM system of FIG. 3.

FIGS. 5A-5F are schematic diagrams illustrating an example visualization of a transseptal puncture performed with the example electrosurgical system of FIG. 1.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are within the ambit of the present disclosure.

Transseptal punctures are often performed with an electrosurgical device having a crossing member disposed within a lumen of a delivery component. The delivery component is applied against a selected location on the atrial septum. The crossing member is extended from the distal tip of the delivery component long enough to tent the tissue and create an opening. Some electrosurgical devices include a crossing member that can only extend an enforced maximum length from the tip of the delivery component. The enforced maximum length is generally related to a distance long enough to tent the tissue and to create the opening, and thus the enforced maximum length may only be a few millimeters. A clinician employing such an electrosurgical device can readily extend the crossing member to the enforced maximum length to efficiently puncture the atrial septum. Some crossing members, however, such as crossing members that are configured as multifunction guidewires, do not have an enforced maximum length of protrusion from the delivery component selected with relation to a distance used to tent tissue and puncture an opening. For instance, crossing members that can be used as multifunction guidewires include a distal tip extendable from the delivery component distal end such that the delivery component is retractable from the patient over the crossing member with the crossing member distal tip disposed within the heart. The multifunction guidewires can be fully extended from the tip of the delivery component. In the case of multifunction guidewires, for instance, protruding the crossing member from the delivery component can be a challenge as users of such devices can inadvertently over-extend the crossing member from the distal tip of the delivery component.

The disclosed system employs a mapping system having a tracking component to detect a position of the electrosurgical device within the heart such as the position of the delivery component and crossing member. The tracking component determines a distance between the crossing member and the delivery component. The mapping system generates an alert if the crossing member is extended from the delivery component at a specified protrusion spacing.

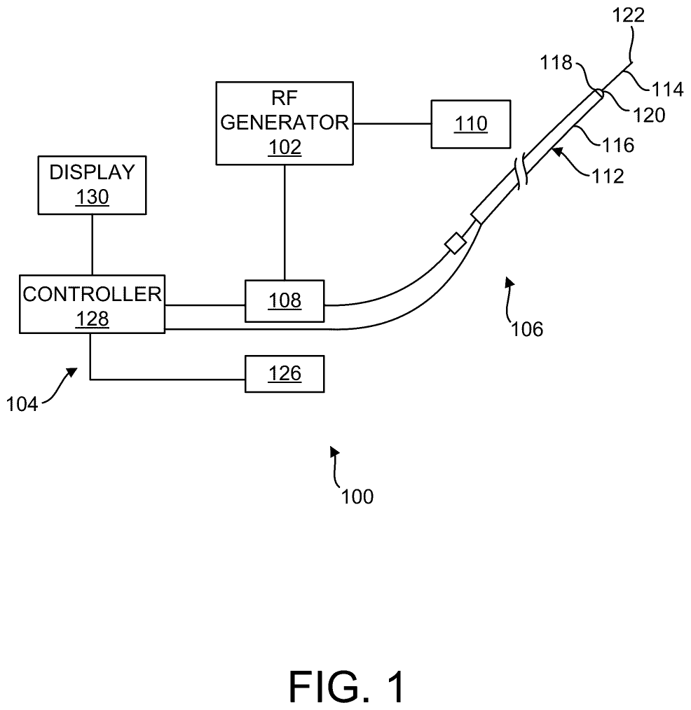

FIG. 1 illustrates an embodiment of an electrosurgical system 100 to facilitate vascular access to a heart and provide catheter positioning within cardiac anatomy. The embodiment of the medical system 100 includes an electrosurgical generator 102, electroanatomical mapping (EAM) system 104, and an electrosurgical device 106. In the illustration, the electrosurgical device 106 is electrically coupled to the electrosurgical generator 102 and the EAM system 104 via a multimode extension cable 108. The electrosurgical generator 102 is configured to provide a source of energy, such as radiofrequency (RF) energy to the electrosurgical device 106 via the cable 108. In some embodiments, the system 100 includes a ground pad electrode, or indifferent (dispersive) patch electrode 110 electrically coupled to the generator 102 for use with the electrosurgical device 106 in a monopolar configuration. In some embodiments, the electrosurgical device 106 is implemented in a bipolar configuration without an indifferent patch electrode. The electrosurgical device 106 includes a delivery component 112 and a crossing member 114. The delivery component 112 includes an elongated shaft 116 having a shaft distal tip 118. The elongated shaft 116 defines a lumen 120. The crossing member 114 is adapted to be disposed within the lumen 120 and coupled to the RF energy source. The crossing member 114 includes a crossing member distal tip 122 extendable from the shaft distal tip 118 of the delivery component 112. The electrosurgical device 106 includes a plurality of electrodes that are electrically coupled to the EAM system 104 such as via cable 108. The illustrated EAM system 104 includes a localization field generator 126, a mapping and navigation controller 128, and a display device 130. The EAM system 104 generates high-fidelity three-dimensional anatomical and electro-anatomical maps of the cardiac chambers of interest. The EAM system 104 includes a tracking component that is operable to track the locations of the various components of the electrosurgical device 106 such as with respect to the structures of the heart or with respect to each other. In one embodiment, the cable 108 can be configured to a mapping setting to electrically couple the EAM system 104 to the electrosurgical device 106 and allow the EAM system 104 to track the location of the electrosurgical device 106. In the embodiment, the cable 108 can be configured in a puncture setting to electrically couple the generator 102 to the electrosurgical device 106 and apply RF energy from the generator 102 to the electrosurgical device 106. The system 100 can be implemented in conjunction with an additional visualization system such as a fluoroscopic visualization system employing a C-arm, or an echocardiogram system such as an intracardiac echocardiography (ICE) system a transesophageal electrocardiography (TEE) system (not shown).

The electrosurgical generator 102 is configured to provide the source of RF energy to the electrosurgical device 106 for a puncture operation. The generator 102 is electrically coupled via cables to the electrosurgical device 106 and patch electrode 110. During a monopolar punction operation of electrosurgical generator 102, a first electrode, often referred to as the active electrode, is provided with the electrosurgical device 106 while a second electrode, such as patch electrode 110, is typically located on the back, buttocks, upper leg, or other suitable anatomical location of the patient during surgery. In such a configuration, the patch electrode 110 is often referred to as a patient return electrode. The cable 108, which can include a switch, can be configured in a puncture setting to electrically couple the electrosurgical generator with the electrosurgical device 106 via cable 108. An electrical circuit of RF energy is formed between the active electrode and the patch electrode 110 through the patient, which is used to puncture tissue at the active electrode. For example, RF energy for a puncture function in a monopolar mode may be provided at a relatively low voltage and a continuous current (100% on, or 100% duty cycle). Nominal impedance can range between 300 to 1000 ohms for the cutting function. At a power setting of 90 Watts for cutting, voltage can range from approximately 164 to 300 volts root mean square (RMS). The electrosurgical generator 102 can include a plurality of functions and provide a programmed and custom settings via an interface and be couplable to a suite of electrosurgical tools in addition to the electrosurgical device 106.

The EAM system 104 is operable to track the location of the various components of the electrosurgical device 106, and to generate high-fidelity three-dimensional anatomical and electro-anatomical maps of the heart, including portions of the heart such as cardiac chambers of interest or other structures of interest such as the sinoatrial node or atrioventricular node as visualizations on display 130. In one illustrative example, the EAM system 70 can include the RHYTHMIA™ HDx mapping system marketed by Boston Scientific Corporation. The mapping and navigation controller 128 of the EAM system 104 includes one or more controllers, such as microprocessors or computers, that execute code out of memory to control or perform functional aspects of the EAM system 104.

The EAM system 104 generates a localization field via the field generator 126 to define a localization volume about the heart. The cable 108 can be switched to a mapping setting, and a location sensor or sensing element on a tracked device, such as electrodes on the electrosurgical device 106, generate an output that is provided to the EAM system 104. The signals from the electrosurgical device 106 are processed by the mapping and navigation controller 128 to track the location of the electrodes, and consequently, the corresponding components of the electrosurgical device 106, within the localization volume. In one embodiment, impedance tracking methodologies are be employed to track the locations of the electrosurgical device 106. For instance, the localization field is an electric field generated, for example, by an external field generator arrangement of the field generator 126, such as surface electrodes, and is received by intra-body or intra-cardiac devices, such as an intracardiac catheter, or both. In these examples, the location sensing elements can constitute electrodes on the tracked devices that generate outputs received and processed by the mapping and navigation controller 128 to track the location of the various location sensing electrodes within the localization volume. In another embodiment, the device tracking is accomplished using magnetic tracking techniques, in which the field generator is a magnetic field generator that generates a magnetic field defining the localization volume, and location sensors on the tracked devices are magnetic field sensors. The EAM system 104 can be equipped for both magnetic and impedance tracking capabilities. In such examples, impedance tracking accuracy can, in some instances be enhanced by first creating a map of the electric field induced by the electric field generator within the cardiac chamber of interest using a probe equipped with a magnetic location sensor.

Regardless of the tracking methodology employed, the EAM system 104 utilizes the location information for the various tracked devices, along with cardiac electrical activity acquired by, for example, another catheter or probe equipped with sensing electrodes, to generate, and display via the display 130, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 104 can generate a graphical representation of the various tracked devices within the geometric anatomical map or the electro-anatomical map on display 130.

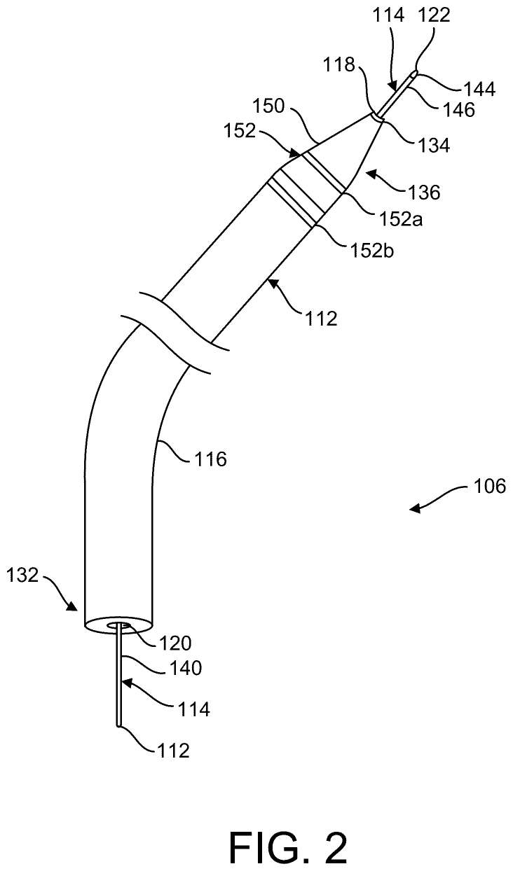

FIG. 2 illustrates electrosurgical device 106 configured to be coupled to the RF energy source and including the delivery component 112 and the crossing member 114. The delivery component 112 includes the elongated shaft 116 defining the longitudinally extending lumen 120 having a shaft proximal end 132 and the shaft distal tip 118 on shaft distal portion 136. The shaft distal tip 118 includes a delivery component electrode, or EAM electrode 134 in the illustration. The coaxial crossing member 114 is adapted to be disposed within the lumen 120 and coupled to the RF energy source. The crossing member includes a crossing member shaft 140 with a crossing member proximal end 142 and the crossing member distal tip 122. The crossing member distal tip 122 includes a crossing member electrode 144 adapted to deliver the RF energy.

In the illustrated example, the crossing member 114 is configured as a multifunction conductive guidewire. For instance, the crossing member 114 can be used, without exchanges, as a guidewire, a transseptal puncture device, or as an exchange rail for delivering therapy sheaths. Such embodiments provide efficiencies to medical procedures as the crossing member 114 performs multiple functions and reduces the amount of device exchanges in the medical procedure. The crossing member 114 is sufficiently thin and flexible to access the various chambers of the heart. The crossing member distal tip 122 is operable to deliver RF energy to puncture the atrial septum from the right atrium through which puncture a distal section 146 of the crossing member shaft 140 is advanced. Once advanced through the puncture site and sufficiently extended from within the shaft distal tip 118 of the delivery component 112, the distal section 146 is biased to form a coil for anchoring the crossing member 114 beyond the puncture site. The delivery component 112 is retractable from the patient over the multifunction guidewire crossing member 114 with the crossing member distal tip 122 disposed within the heart. The multifunction guidewire crossing member 114 can also support the installation of tubular members or other catheters and for advancing other devices within the heart.

The crossing member 114 is configured to conduct an electrical signal, from the proximal end 142 to the exposed electrode 144. The exposed electrode 144 is configured to apply the RF energy to puncture tissue. For example, the exposed electrode 144 is electrically coupled to the electrosurgical generator 102 via the cable 108 switched to the puncturing setting. Additionally, the exposed electrode 144 is electrically coupled to the EAM system 104 via the cable 108 switched to the mapping setting and through a pin connector of the EAM system so that electrical signals can be communicated between the EAM system 104 and the exposed electrode 144. In some instances, the relative location of the exposed electrode 144 can be determined by the EAM system 104 such as when the exposed electrode 144 is extended from the shaft distal tip 118 of the delivery component 112.

In some embodiments, the delivery component is 112 is configured as a dilator or dilator/sheath assembly. For instance, the elongated shaft 116 includes a distal tapered portion 160 with an enlargement of cross-sectional area with respect to the shaft distal tip 118. As the distal tapered portion 150 is passed through an aperture from the shaft distal tip 118, the enlargement of cross-sectional area dilates the aperture. The dilator can be configured as a straight dilator, as illustrated, or a curved dilator. The elongated shaft 116 can be made from various materials including insulative materials such as high-density polyethylene (HDPE). The EAM electrode 134 is located on the distal tapered portion 150 on the shaft distal tip 118. The EAM electrode 134 is positioned to allow for a determination of the shaft distal tip 118, such as directly mounted to the shaft distal tip 118. In some embodiments, the EAM electrode 134 is positioned to allow for an indirect determination of the shaft distal tip 118, such proximally from the shaft distal tip 118 and in which the EAM system 104 performs an extrapolation to determine the location of the shaft distal tip 118 based on the location of the of the EAM electrode 134 on the distal tapered portion 150 or the elongated shaft 116. In the illustrated example, the EAM electrode 134 is annular and forms a ring about the shaft distal tip 118. The EAM electrode 134 in the illustration is profiled to match the taper of the distal tapered portion 150. The EAM electrode 134 is electrically coupled to an electrical conductor (not shown) that extends along the elongated shaft 116 and is electrically coupled to the EAM system 104, such as via a pin connector of the EAM system. Electrical signals can be communicated between the EAM system 104 and the EAM electrode 134 so that the relative location of the EAM electrode 134, such as with respect to structures of the heart, can be determined by the EAM system 104. In some embodiments, the EAM system 104 can determine the relative location of the EAM electrode 134 with respect to the location of the exposed electrode 144 on the crossing member 114 such as when the exposed electrode 144 is extended from the shaft distal tip 118 of the delivery component 112, e.g., when the exposed electrode 144 is distal to the EAM electrode 134.

In some embodiments, the shaft distal portion 136 of the delivery component 112 can include an additional one or more electrodes, such as an EAM orientation electrode 152. For instances, an EAM orientation electrode 152a can be positioned on the distal tapered portion 150 of a dilator and proximal to the EAM electrode 134 on the shaft distal tip 118, an EAM orientation electrode 152b can be positioned on a sheath of a dilator/sheath assembly and proximal to the EAM electrode 134 on the shaft distal tip 118, or both EAM orientation electrodes 152a, 152b can be included on the shaft distal portion 136. The EAM orientation electrode 152 is positioned on the delivery component 112 such as along the lumen 120 so that the expected position of the crossing member 114 within the delivery component 112 can be ascertained. In some embodiments, the EAM orientation electrode 152 is configured in a fixed spacing with relationship to the EAM electrode 134 on the shaft distal tip 118. In the illustrated example, the EAM orientation electrode 152 is annular and forms a ring about the shaft distal portion 136 such as the dilator tip in electrode 152a or sheath tip in electrode 152b. The EAM orientation electrode 152 is electrically coupled to an electrical conductor (not shown) that extends along the elongated shaft 116 and is electrically coupled to the EAM system 104, such as via a pin connector of the EAM system. Electrical signals can be communicated between the EAM system 104 and the EAM orientation electrode 152 so that the EAM system 104 can determine the relative location of the EAM orientation electrode 152 with respect to the location of the EAM electrode 134 on the shaft distal tip and other EAM orientation electrodes.

In an anticipated use of the system 100, the electrosurgical device 106 is coupled to the RF generator 102 and the EAM system 104 such as via cable 108. If the electrosurgical device 106 is to be configured in a monopolar mode, the patch electrode 110 is coupled to the patient. The RF generator 102 can be set to a puncture mode, such as an energy output of approximately 10 watts. The field generator 126 can be coupled to the patient. In some examples, femoral access is obtained via a conventional percutaneous needle, and a guidewire is inserted into the vasculature and advanced to the superior vena cava. In some embodiments, the crossing member 114 can be used as the guidewire. The shaft distal tip 118 of the delivery component 112 is advanced over the proximal end of the guidewire, and the distal tapered portion 150 of the delivery component shaft 116 is advanced over the guidewire to the superior vena cava. Under visualization, such as via tracking with the EAM system 104 after mapping the heart, or at least the right-sided anatomy of the heart, the distal tapered portion 150 is moved from the superior vena cava to the right atrial septum and then to the fossa ovalis of the heart. Once the delivery component distal tip 118 is confirmed at the fossa ovalis, such as via visualization of the EAM electrode 134 on the EAM system 104, the electrode 144 of the crossing member 114 is advanced from the shaft distal tip 118 of the delivery component 112. In one example, the exposed electrode 144 of the crossing member 114 is extended a few millimeters from the shaft distal tip 118 to tent the heart tissue and apply RF energy. In general, the crossing member 114 is extended longitudinally for several millimeters prior to the distal section 146 curving to assume a J-tip or pigtail shape and deflecting away from the atrial septum. Forward pressure is applied to the electrosurgical device 106 and the crossing member 114 is actuated to apply the RF energy to the electrode 144 and puncture the fossa ovalis. The crossing member 114 can be advanced into the left atrium of the heart and anchored. In one example of the delivery component 112 including a distal tapered portion 150 of a dilator, the distal tapered portion 150 is advanced into the puncture site to expand the aperture.

Some electrosurgical devices include a puncture device such as an electrode or mechanical needle that can only extend an enforced maximum length from the tip of the delivery component. Such enforced maximum length is generally related to a distance enough to tent the tissue and generate a puncture in the fossa ovalis. For example, some devices provide for the crossing member to extend up to two or four millimeters from the tip of the delivery component. A clinician employing such a device can readily extend the crossing member to the enforced maximum length to puncture the atrial septum.

Some crossing members, however, do not have an enforced maximum length of protrusion from the delivery component or have an enforced maximum length of protrusion much longer than used to tent and puncture tissue. For instance, crossing members that can be used as guidewires include a distal tip extendable from the delivery component distal end such that the delivery component is retractable from the patient over the crossing member with the crossing member distal tip disposed within the heart. Users of such devices can inadvertently over-extend the crossing member from the distal tip of the delivery component during the tenting phase of transseptal puncture, particularly if the user is only familiar with enforced maximum length type electrosurgical device.

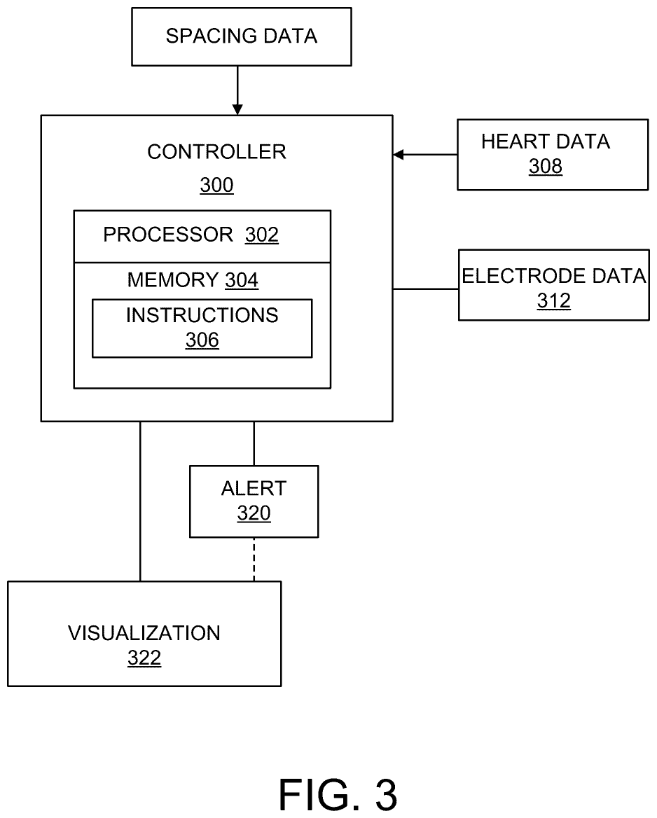

FIG. 3 illustrates an example controller 300 that can be implemented with EAM system 104 in the electrosurgical system 100. In one embodiment, controller 300 can be implemented with mapping and navigation controller 128 of the EAM system 104. The controller 300 is implemented to detect a position of the electrosurgical device within the heart based on the delivery component and crossing member electrodes and determine whether the crossing member electrode is extended from the delivery component electrode at a specified protrusion spacing via tracking extension of the crossing member electrode from the distal tip electrode. The controller generates a notice, such as an alert on a visualization, if the crossing member electrode is extended from the delivery component electrode at the specified protrusion spacing. The controller 300 can include a processor 302 and a memory 304. The memory 304 stores processor executable instructions 306. For instance, the processor executable instructions 306 can be in the form of a program, such as a computer program or application. The processor 302 is implemented to execute the instructions 306 that configure the controller 300. In some embodiments, the controller 300 is implemented as a computing device such as a laptop computer, a workstation, a desktop computer, a tablet, or a smartphone. In some embodiments, the controller 300 includes additional components such as a display, a touchscreen, speakers or other output devices, a keyboard or other input devices, or communication circuitry such as computer network adapters.

In some embodiments, the processor 302 includes a plurality of main processing cores to run an operating system and perform general-purpose tasks on an integrated circuit. The processor 302 can also include built-in logic or a programmable functional unit, also on the same integrated circuit with an instruction-set architecture. In additional to multiple general-purpose, main processing cores and the application processing unit, the controller 300 in embodiments includes other devices or circuits such as graphics processing units or neural network processing units, which may include heterogeneous or homogenous instruction set architectures with the main processing cores.

Memory 304 is an example of computer storage media. Computer storage media includes RAM, ROM, EEPROM, flash memory or other memory technology, CD-ROM, digital versatile discs (DVD) or other optical storage, magnetic cassettes, magnetic tape, magnetic disk storage or other magnetic storage devices, USB flash drive, flash memory card, or other flash storage devices, or other storage medium that can be used to store the desired information and that can be accessed by the processor 302. Any such computer storage media may be part of the controller 300 and implemented as memory 304. Memory 304 is a non-transitory, processor readable memory device. Accordingly, a propagating signal by itself does not qualify as storage media or memory 304.

The controller 300 is configured to receive inputs or information from the electrosurgical system 100, such as inputs from the electrosurgical device 106 and inputs from the EAM system 104 including the mapping and navigation controller 128, for storage in memory 304 and use by the instructions 306. In some embodiments, the controller 300 receives an input representative of the anatomical map of the heart, or heart map data 308, which heart map data 308 can include the data regarding representations of the geometric anatomical map of the heart and the electro-anatomical map of the heart, such as from the EAM system 104 or previous scans or images of the heart. Additionally, the controller 300 receives protrusion spacing data 310, such as a user-selected amount of protrusion of the exposed electrode 144 of the crossing member 114 from the distal tip 118 of the delivery component 112. In one embodiment, the spacing data 310 can be entered via an input device such as a keyboard in response to a prompt or field presented by the program in preparation for a procedure. In other embodiments, the protrusion spacing data 310 can be determined from an input of procedure parameter data, which can include information regarding the electrosurgical device employed in the procedure, and then determined from such information as heart structure data. Further, the controller 300 received electrosurgical device location data 312, such as signals from the exposed electrode 144 on the crossing member 114 and the EAM electrode 134 on the delivery component 112.

The controller 300 is configured to generate an alert 320 if the crossing member electrode is extended from the delivery component electrode is at the specified protrusion spacing according to the spacing data 310. The alert 320 can be presented as notification on an output device such as a speaker or a display 130 of the EAM system 104. In the illustrated embodiment, the alert 320 can be part of a visualization 322 that can notify a user when the crossing member electrode is extended from the delivery component electrode at the specified protrusion spacing according to the spacing data 310. The visualization 322 can be implemented as providing tracking data of the EAM system 104.

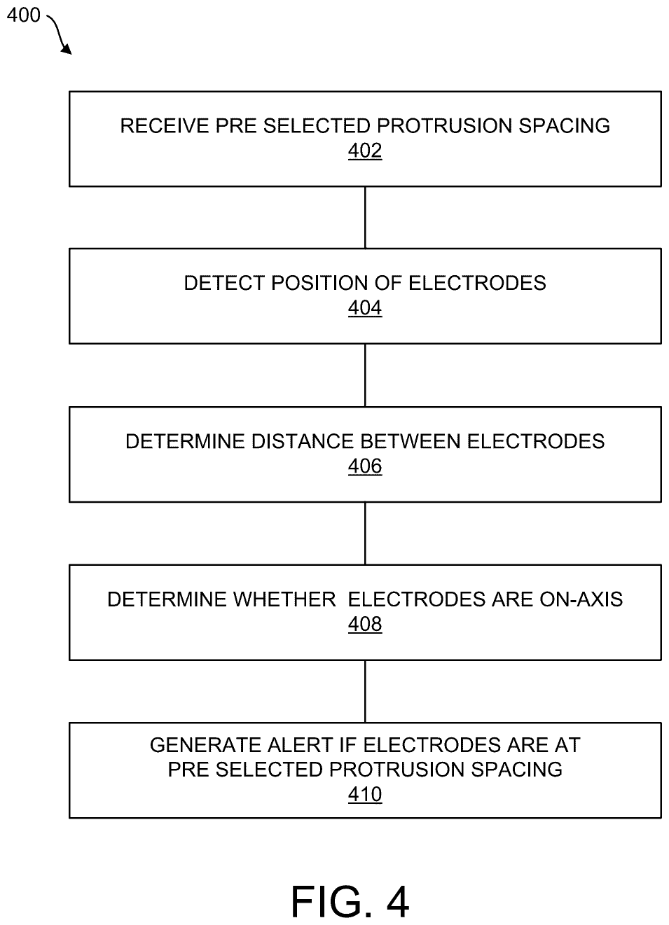

FIG. 4 illustrates a process 400 implemented in the controller 300 of EAM system 104, such as via a tracking component of the EAM system and after the heart has been mapped. In embodiments, the process 400 is a computerized process. In one embodiment, process 300 is implemented as set of processor-executable instructions, such as instructions 306, stored in a non-transitory memory, such as memory 304 to be executed by a processor 302 to configure controller 300. The instructions 306 to implement process 400 can be configured to receive information, such as to retrieve from memory 404 heart map data 308, spacing data 310, and electrosurgical device location data 312. Further, the instructions 306 to implement process 400 can be configured to generate a visualization, such as visualization 320 on a display of graphical representations on display device 130.

Process 400 includes receiving a specified protrusion spacing at 402. The protrusion spacing is a distance between the electrode 144 of the crossing member 114 advanced from the shaft distal tip 118 of the delivery component 112. As the electrode 144 is movable with respect to the shaft distal tip 118, protrusion spacing can vary during a procedure as the electrode 114 is moved with respect to the shaft distal tip 118. The specified protrusion spacing, however, is the distance the electrode 144 of the crossing member 114 is advanced from the shaft distal tip 118 of the delivery component 112 to tent and puncture the heart structure. The specified protrusion spacing is set during use such as in preparation for a procedure or during a procedure. For instance, a user can input a specified protrusion spacing into the EAM system 104 prior to use or after mapping and consideration of the atrial septum. In another instance, the specified protrusion spacing is preprogrammed into the EAM system 104 or generated by the EAM system 104 during use. In one embodiment, the EAM system 104 can present a prompt on a graphical display to request user inputs regarding electrode spacing. For instance, the EAM system 104 can present a prompt for a protrusion spacing to tent and puncture the heart structure, and the user response to this prompt can become the specified protrusion spacing if, for instance, the input data is a selected data type and within a selected range of acceptable protrusion spacings. If not, the EAM system 104 can issue an error and present the prompt again. Alternatively, the EAM system 104 can present a drop-down menu of available specified protrusion spacings, and the user can select one of the available specified protrusion spacings. In another embodiment, the EAM system 104 can present prompts for a user to input a set of surgical parameters related to the electrosurgical device 106 and the heart. Surgical parameters can include the model of the delivery component 112 and crossing member 114, information related to the thickness of the atrial septum, and historical or trend data from previous surgeries. The EAM system 104 determines the specified protrusion spacing from a set of received surgical parameters.

The process also includes detecting a position of the delivery component electrode and the crossing member electrode at 404. For example, the location of the delivery component electrode is detected with respect to the location of the crossing member electrode with the EAM system 104. The detected positions of the delivery component electrode and the crossing member electrode can be presented on a graphical display. In one embodiment, the EAM system 104 generates a graphical representation of the heart, such as part of the heart anatomy. The positions of the delivery component electrode 134 and the crossing member electrode 144 are superimposed on the graphical representation of the heart to present the locations of the electrodes 134, 144 with respect to each other and with respect to structures of the heart. In some embodiments, the position of the crossing member electrode 144 is not detectable when the crossing member electrode 144 is within the shaft 116 of the delivery component 112, such as proximal to the shaft distal tip 118. In such embodiments, the position of the crossing member electrode 144 is detectable if the crossing member electrode 144 has been extended from the shaft distal tip 118. If in these embodiments the crossing member electrode is disposed within the shaft 116, the EAM system 104 can detect the position of the delivery component electrode 134 and present the location of the delivery component electrode 134 on the graphical representation of the heart and not the position of the crossing member electrode 144. In some embodiments, the EAM system 104 can present whether the crossing member electrode 144 has been determined to be extended from the shaft distal tip 118 on the graphical display.

A distance between the crossing member electrode and the delivery component electrode is determined at 406. For instance, the distance between the crossing member electrode 144 from the delivery component electrode 134 is determined via tracking the crossing member electrode 144 as it is extended from the shaft distal tip 118 with the EAM system 104. As the crossing member 114 is movable with respect to the delivery component 112, the distance between the crossing member electrode 144 and the delivery component electrode 134 will vary. In some embodiments, the distance between the crossing member electrode 144 and the delivery component electrode 134 is determined in real time. In some embodiments, the EAM system 104 can present the actual or relative real time distance between the crossing member electrode 144 and the delivery component electrode 134 on the graphical display.

Embodiments in which the EAM orientation electrode 152 is used in combination with the EAM electrode 134 can include a feature to determine whether the crossing member electrode 144 is on axis at 408. For instance, if the crossing member electrode 134 is on an axis defined by the EAM electrode 134 and the EAM orientation electrode 152, the crossing member electrode 134 is configured to tent and puncture the tissue. If the crossing member electrode 134, however, is off the axis defined by the EAM electrode and the EAM orientation electrode 152, the crossing member 114 is likely in a J-tip configuration or coiled and is not configured to tent and puncture the tissue. In such as case, the crossing member 114 is overextended from the shaft distal tip 118 and can be retracted in the delivery component 112. An axis is determined a defined by a line through the detected position of the EAM electrode 134 and the detected position or positions of the one or more EAM orientation electrodes 152. If the detected position of the delivery component electrode 134 is determined to be on the defined line, the crossing member electrode 134 is determined to be on axis at 408. If the detected position of the delivery component electrode 134 is determined not to be on the defined line, the crossing member electrode 134 is determined to be off axis at 408. In some embodiments, the defined line can be detected in three dimensions, and the determination of whether the crossing member electrode 134 is on the defined line is performed in three dimensions. In embodiments, the determination of whether the crossing member electrode is on axis is based on a congruence of the position of the crossing member electrode and the line defined by the EAM electrode 144 and the EAM orientation electrode 152.

An alert is generated if the crossing member electrode is extended from the delivery component electrode at a specified protrusion spacing at 410. In embodiments, the alert is generated with the EAM system 104 based on a congruence of the position of the delivery component electrode 134 and the crossing member electrode 144 and the specified protrusion spacing. In one embodiment, the variable distance between the delivery component electrode 134 and the crossing member electrode 144 is tracked and compared to the specified protrusion spacing. If the variable distance is congruent with the specified protrusion spacing, an alert is generated at 410. In another embodiment, the EAM system 104 seeks a pair of electrodes 134, 144 at a distance congruent with the specified protrusion spacing. No alert is issued if the electrodes 134, 144 are spaced apart at a distance outside of range of acceptable error in the specified protrusion spacing. If the electrodes 134, 144 are detected at a distance apart that is congruent with the specified protrusion spacing, an alert is generated at 410.

In embodiments that determine whether the crossing member electrode is on axis at 408, an alert is generated if the crossing member electrode is extended from the delivery component electrode at a specified protrusion spacing at 410 and the crossing member is determined to be on axis. In some embodiments, if the electrodes 134, 144 are detected at a distance apart that is congruent with the specified protrusion spacing, then the EAM system 104 determines whether the crossing member electrode 152 in on axis. If the crossing member electrode is determined to be off axis in this embodiment, then no alert is issued at 410 even if the variable distance is congruent with the specified protrusion spacing.

The alert at 410 can take one or more of several forms. Embodiments of the alert include a tone or computerized spoken phrase over a speaker on the EAM system 104, a visual indication of the display device 130 or the electrosurgical device 106, a haptic response on the electrosurgical device 106. In one embodiment, the alert includes highlight a graphical illustration of the crossing member electrode extended from the delivery component electrode superimposed on a graphical representation of the heart for the display device 130.

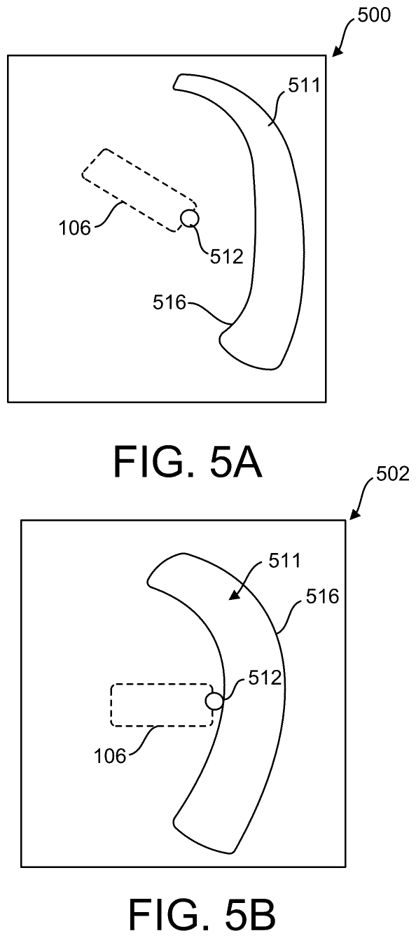

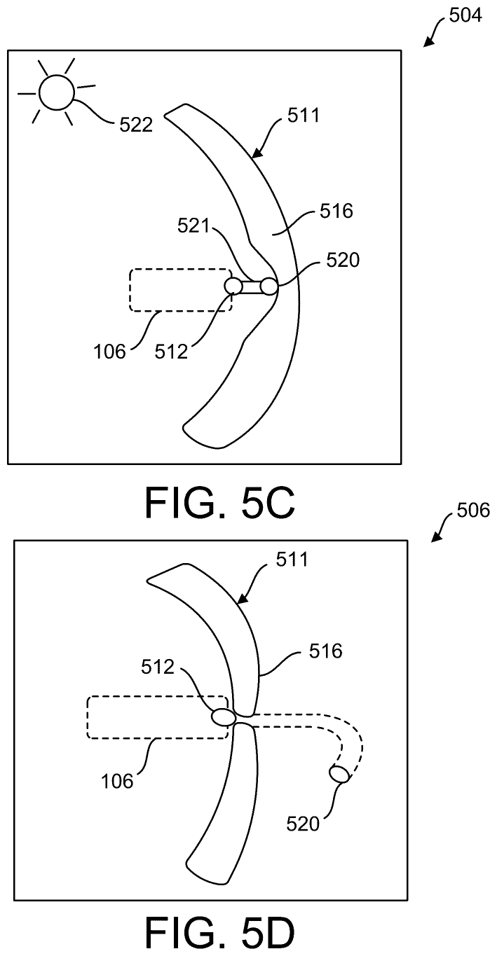

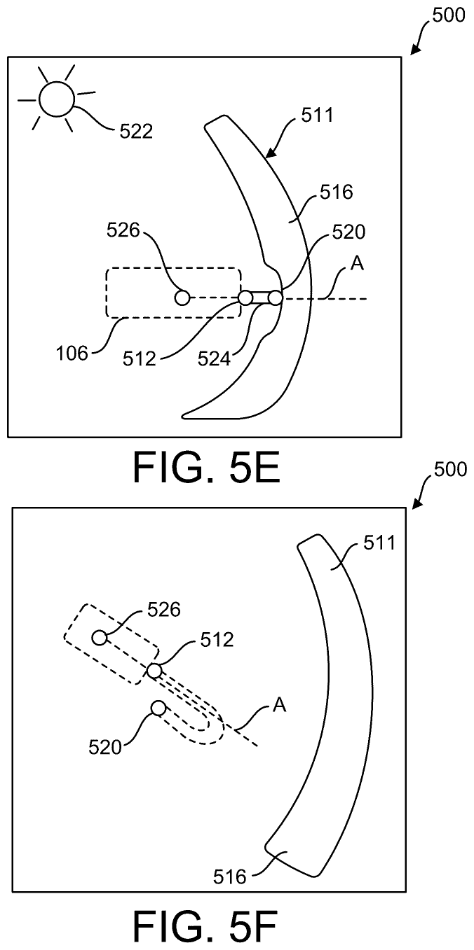

FIGS. 5A-5F illustrate schematic of visualizations 500, 502, 504, 506, 508, 510 respectively, generated on display 130 of EAM system 104 in implementing the process 400 of the electrosurgical device 106 during a transseptal puncture procedure. In the example visualizations, the heart has been mapped, and the graphical representations of the electrosurgical device 106 is superimposed on the graphical representation of the heart 511.

For the visualization 500 of FIG. 5A, the electrosurgical device 106 (illustrated in phantom for reference) has entered the right atrium and is approaching the atrial septum. In the visualization 500, the relative position of the delivery component electrode 134 is superimposed on the graphical representation of the right atrium of the heart 511 as point 512. The relative position of the crossing member electrode 144 is disposed within the shaft and is not presented on the visualization 500. In one embodiment, the electrodes 134, 144 may be distinguished from one another via different colors, blinking lights, or other indica.

For the visualization 502 of FIG. 5B, the shaft distal tip 118 of the electrosurgical device 106 (illustrated in phantom for reference) is disposed against the atrial septum in the right atrium. In the visualization 502, the relative position of the delivery component electrode 134 is superimposed on the graphical representation of the heart 511 as point 512, which is disposed against the graphical representation of the atrial septum 516. The relative position of the crossing member electrode 144 is again disposed within the shaft and is not presented on the visualization 500.

For the visualization 504 of FIG. 5C, the shaft distal tip 118 of the electrosurgical device 106 (illustrated in phantom for reference) is disposed against the atrial septum, and the crossing member electrode 144 is extended at the selected protrusion spacing received by process 400 to tent the atrial septum. In this example, the electrosurgical device 106 is prepared to puncture the atrial septum with RF energy. In the visualization 504, the relative position of the delivery component electrode 134 is superimposed on the graphical representation of the heart 511 as point 512, which is proximal to the graphical representation of the atrial septum 516. The relative position of the crossing member electrode 144 is superimposed on the graphical representation of the heart 511 as point 520, which is disposed against to the graphical representation of the atrial septum 516. As the crossing member electrode 144 is extended from the delivery component electrode 134 at the specified protrusion spacing as determined with the EAM system 104 implementing process 400, the visualization 504 presents an alert. For the alert in visualization 504, a blinking light 522 appears. Additionally, in the embodiment, the graphical representation of the crossing member electrode 520 extended from the graphical representation of the delivery component electrode 514 superimposed on the graphical representation of the heart 511 is highlighted, such as with bright line 524. Other examples of highlighting the graphical representations, include the use of different colors or blinking lights.

For the visualization 506 of FIG. 5D, the shaft distal tip 118 of the electrosurgical device 106 (illustrated in phantom for reference) is disposed proximate the atrial septum, and the crossing member electrode 144 is extended at into the left atrium at a distance greater than the selected protrusion spacing received by process 400. In this example, the electrosurgical device 106 has punctured the atrial septum with RF energy and the crossing member 114 is prepared to anchor in the left atrium. In the visualization 506, the relative position of the delivery component electrode 134 is superimposed on the graphical representation of the heart 511 as point 512, which is proximal to the graphical representation of the atrial septum 516. The relative position of the crossing member electrode 144 is superimposed on the graphical representation of the heart 511 as point 520, which is disposed in the graphical representation distal to the atrial septum 516. As the crossing member electrode 144 is extended from the delivery component electrode 134 at a distance greater than and not congruent with the specified protrusion spacing as determined with the EAM system 104 implementing process 400, the visualization 504 does not present an alert in this example.

For the visualizations 508 of FIG. 5E and 510 of FIG. 5F, an EAM orientation electrode is used to determine whether the crossing member electrode is on axis as implemented in 408 of process 400.

For the visualization 508 of FIG. 5E, the shaft distal tip 118 of the electrosurgical device 106 (illustrated in phantom for reference) is disposed against the atrial septum, and the crossing member electrode 144 is extended at the selected protrusion spacing received by process 400 to tent the atrial septum. In this example, the electrosurgical device 106 is prepared to puncture the atrial septum with RF energy. In the visualization 508, the relative position of the delivery component electrode 134 is superimposed on the graphical representation of the heart 511 as point 512, which is proximal to the graphical representation of the atrial septum 516. The relative position of the crossing member electrode 144 is superimposed on the graphical representation of the heart 511 as point 520, which is disposed against to the graphical representation of the atrial septum 516. The relative position of orientation electrode 152 is superimposed on the graphical representation of the heart 511 as point 526. A line extending through the delivery component electrode and orientation electrode is defined as axis A. In embodiments, the axis A is depicted on the visualization 508. As the crossing member electrode 144 is extended from the delivery component electrode 134 at the specified protrusion spacing and on axis A as determined with the EAM system 104 implementing process 400, the visualization 504 presents an alert. For the alert in visualization 504, a blinking light 522 appears. Additionally, in the embodiment, the graphical representation of the crossing member electrode 520 extended from the graphical representation of the delivery component electrode 514 superimposed on the graphical representation of the heart 511 is highlighted, such as with bright line 524. Other examples of highlighting the graphical representations, include the use of different colors or blinking lights.

For the visualization 510 of FIG. 5F, the shaft distal tip 118 of the electrosurgical device 106 (illustrated in phantom for reference) is approaching the atrial septum, and the crossing member electrode 144 is extended at the selected protrusion spacing received by process 400 to tent the atrial septum. In this example, however, the electrosurgical device 106 is not prepared to puncture the atrial septum with RF energy because the crossing member is overextended from the delivery component. The overextended crossing member is in a J-tip configuration within the atrium, but the spacing between the crossing member electrode 144 and the delivery component electrode 134 is at the selected protrusion spacing. In the visualization 510, the relative position of the delivery component electrode 134 is superimposed on the graphical representation of the heart 511 as point 512, which is proximal to the graphical representation of the atrial septum 516. The relative position of the crossing member electrode 144 is superimposed on the graphical representation of the heart 511 as point 520. The relative position of orientation electrode 152 is superimposed on the graphical representation of the heart 511 as point 526. A line extending through the delivery component electrode and orientation electrode is defined as axis A. In embodiments, the axis A is depicted on the visualization 508. As the crossing member electrode 144 is extended from the delivery component electrode 134 at the specified protrusion spacing but off axis A, or not on axis A, as determined with the EAM system 104 implementing 408 of process 400, the visualization 504 does not present an alert that the crossing device is configured to tent and puncture the atrial septum.

Various modifications and additions can be made to the exemplary embodiments discussed without departing from the scope of the present disclosure. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A transeptal surgical system configured to puncture an atrial septum within a heart of a patient, the transseptal surgical system comprising:
an electrosurgical device configured to couple to a radiofrequency (RF) energy source, the electrosurgical device comprising:
a delivery component having an elongate shaft defining a longitudinally extending lumen and a delivery component distal tip having a delivery component electrode, and
a crossing member adapted to be disposed within the lumen and coupled to the RF energy source, the crossing member having a crossing member distal tip extendable from the lumen, the crossing member distal tip having a crossing member electrode adapted to deliver the RF energy; and
an electroanatomical mapping system coupled to the electrosurgical device, the electroanatomical mapping system having a tracking component configured to:
detect a position of the delivery component electrode and the crossing member electrode,
determine a distance between the crossing member electrode and the delivery component electrode, and
generate an alert if the crossing member electrode is extended from the delivery component electrode at a specified protrusion spacing.

2. The transeptal surgical system of claim 1, wherein the delivery component is a dilator having a tapered distal portion, the tapered distal portion including the delivery component distal tip.

3. The transeptal surgical system of claim 1, and further comprising an RF generator to provide the RF energy source.

4. The transseptal surgical system of claim 3, and further comprising a switch mechanically coupled to the electrosurgical device, the RF generator, and the electroanatomical mapping system, the switch configured to selectively electrically couple the electrosurgical device to one of the RF generator and the electroanatomical mapping system.

5. The transseptal surgical system of claim 4, wherein the switch is configured to selectively electrically couple the crossing member electrode to one of the RF generator and the electroanatomical mapping system.

6. The transeptal surgical system of claim 1, wherein the crossing member is adapted to deliver the RF energy in a monopolar mode.

7. The transeptal surgical system of claim 1, wherein the crossing member is configured as a guidewire.

8. The transeptal surgical system of claim 1, wherein the crossing member distal tip is extendable from the delivery component distal tip such that the delivery component is fully retractable over the crossing member.

9. The transeptal surgical system of claim 1, wherein the delivery component includes an orientation electrode, and the tracking component is further configured to:
detect a position of the orientation electrode;
define an axis of the delivery component to include the position of the delivery component electrode and the orientation electrode; and
generate the alert if the crossing member electrode is extended from the delivery component electrode at the specified protrusion spacing and the
detected position of the crossing member electrode lies on the axis of the delivery component.

10. The transeptal surgical system of claim 9, wherein the orientation electrode is disposed proximal to the delivery component electrode along the elongate shaft.

11. An electroanatomical mapping system for use with an electrosurgical device to guide a surgical puncture of an atrial septum within a heart of a patient, the electrosurgical device comprising a delivery component having an elongate shaft defining a longitudinally extending lumen and a delivery component distal tip having a delivery component electrode, and a crossing member adapted to be disposed within the lumen, the crossing member having a crossing member distal tip extendable from the lumen, the crossing member distal tip having a crossing member electrode adapted to deliver a source of RF energy, the electroanatomical mapping system comprising:
memory to store a set of instructions; and
a processor to execute the set of instructions to:
detect a position of the delivery component electrode and the crossing member electrode,
determine a distance between the crossing member electrode and the delivery component electrode, and
generate an alert if the crossing member electrode is extended from the delivery component electrode at a specified protrusion spacing.

12. The electroanatomical mapping system of claim 11, wherein the set of instructions further includes instructions to receive the specified protrusion spacing from a user input.

13. The electroanatomical mapping system of claim 11, wherein the instructions to generate the alert include instructions to generate the alert based on a congruence of the position of the delivery component electrode and the crossing member electrode and the specified protrusion spacing.

14. The electroanatomical mapping system of claim 11, wherein instructions to detect the position of the delivery component electrode and the crossing member electrode include instructions to superimpose the position of the delivery component electrode and the crossing member electrode on a graphical representation of the heart.

15. The electroanatomical mapping system of claim 14, wherein instructions to generate the alert include instructions to highlight a graphical illustration of the crossing member electrode extended from the delivery component electrode superimposed on the graphical representation of the heart.

16. The electroanatomical mapping system of claim 11, wherein the set of instructions further includes instructions to determine the specified protrusion spacing from a set of received surgical parameters related to the electrosurgical device and the heart.

17. A process to guide an electrosurgical device in a surgical puncture of an atrial septum within a heart of a patient, the electrosurgical device comprising a delivery component having an elongate shaft defining a longitudinally extending lumen and a delivery component distal tip having a delivery component electrode, and a crossing member adapted to be disposed within the lumen, the crossing member having a crossing member distal tip extendable from the lumen, the crossing member distal tip having a crossing member electrode adapted to deliver a source of RF energy, the process comprising:
detecting a position of the delivery component electrode and the crossing member electrode with an electroanatomical mapping system electrically coupled to the electrosurgical device;
determining a distance between the crossing member electrode and the delivery component electrode with the electroanatomical mapping system; and
generating an alert with the electroanatomical mapping system if the crossing member electrode is extended from the delivery component electrode at a specified protrusion spacing.

18. The process of claim 17, wherein generating an alert is based on a congruence of the position of the delivery component electrode and the crossing member electrode and the specified protrusion spacing from a user input.

19. The process of claim 18, and further comprising superimposing the position of the delivery component electrode and the crossing member electrode on a graphical representation of the heart in a display device.

20. The process of claim 19, wherein instructions to generate the alert include instructions to highlight a graphical illustration of the crossing member electrode extended from the delivery component electrode superimposed on the graphical representation of the heart.
